(19) United States
(12) Patent Application Publication
(10) Pub. No.: US 2013/0035262 A1
(43) Pub. Date: Feb. 7, 2013

(54) Integrated Computational Element Analytical Methods for Microorganisms Treated with a Pulsed Light Source

(71) Applicants: Robert P. Freese, Pittsboro, NC (US); Johanna A. Haggstrom, Duncan, OK (US); Jason E. Bryant, Spring, TX (US); Rory D. Daussin, Spring, TX (US)

(72) Inventors: Robert P. Freese, Pittsboro, NC (US); Johanna A. Haggstrom, Duncan, OK (US); Jason E. Bryant, Spring, TX (US); Rory D. Daussin, Spring, TX (US)

(21) Appl. No.: 13/545,324

(22) Filed: Jul. 10, 2012

Related U.S. Application Data
(63) Continuation-in-part of application No. 13/204,294, filed on Aug. 5, 2011.

(51) Int. Cl. C12Q 1/70; C09K 8/02; C09K 8/00; C12Q 1/22

(52) U.S. Cl. 507/101; 435/31; 435/5; 507/201

(57) ABSTRACT

Determining the microorganism load of a substance may be conducted readily using one or more integrated computational elements. By determining a substance's microorganism load, the substance's suitability for a variety of applications may be ascertained. Methods for determining the microorganism load of a substance using one or more integrated computational elements can comprise: providing a substance comprising a plurality of viable microorganisms; exposing the substance to a pulsed light source for a sufficient length of time to form at least some non-viable microorganisms; and determining a microorganism load of the substance using one or more integrated computational elements.

### CROSS-REFERENCE TO RELATED APPLICATIONS

This application is a continuation-in-part of U.S. patent application Ser. No. 13/204,294, filed on Aug. 5, 2011, which is incorporated herein by reference in its entirety.

### BACKGROUND

The present invention generally relates to the monitoring of microorganisms, and, more specifically, to the use of one or more integrated computational elements to determine the effectiveness of a microorganism treatment.

The presence of bacteria and other microorganisms in a substance is often determined after enhancing low levels of biological material to detectable levels. In some cases, an aliquot of the substance can be cultured under conditions that are conducive for growth of a particular biological material. In other cases, nucleic acid amplification techniques, such as polymerase chain reaction (PCR), can be used to increase levels of nucleic acids. Culturing methods, in particular, may sometimes be non-specific, as many different types of microorganisms may grow under the chosen culturing conditions, whereas only certain microorganisms may be of interest for an analysis. Furthermore, both culturing and nucleic acid amplification techniques are often constrained by the timeframe over which they are conducted. PCR techniques, for example, may take several hours or more to produce sufficient nucleic acid quantities for analysis, and culturing may take days to weeks to complete. Methods for real-time or near real-time monitoring of bacteria and other microorganisms are not believed to have yet been developed.

The present inability to monitor bacteria and other microorganisms in a sufficiently rapid manner can have significant ramifications for a variety of commercial and industrial products and processes. For example, due to a limited shelf life, a product (e.g., a foodstuff or pharmaceutical) may have been transported to a store and released for public consumption before product quality testing has been fully completed. By the time a biological contamination has been uncovered, it can oftentimes be too late, as consumers may have already come in contact with the contaminated product. Not only can human health be compromised, but valuable process time, raw materials, and other resources may have been lost by preparing and distributing a contaminated product.

Although biological contamination is a recognizable concern in the food and drug industry, the problem of contamination by bacteria and other microorganisms extends to a much broader array of fields, including those not directly impacting human health. For example and without limitation, biological monitoring of water treatment and wastewater processing streams, including those from refineries, can be of significant interest due to downstream contamination issues. In subterranean operations, biological contamination can reduce production and/or result in biofouling of equipment and wellbore surfaces. In addition, biological contamination on some solid surfaces can lead to structural defects, including corrosion, that ultimately may result in mechanical failure. In short, any industry in which monitoring of biological contamination is of interest could potentially benefit from more rapid detection techniques for biological materials.

Concurrently with monitoring for the presence of biological materials, there often can be an interest in reducing and/or preventing biological contamination within a substance, including on a surface. In some instances, a biocide may be used to slow or stop biological growth. Although biocides may often be effective for addressing biological contamination, their effects can sometimes be slow acting. In addition, at least some members of a population of microorganisms usually survive treatment with a biocide. Another technique that may be used to slow or stop biological growth is irradiation with a source of electromagnetic radiation (e.g., ultraviolet radiation). Continuous-operation ultraviolet radiation sources, particularly mercury vapor ultraviolet radiation sources, are often used for this purpose.

Some bacteria and other microorganisms, instead of being killed outright by continuous-operation ultraviolet radiation sources, may undergo a transformation whereby they are still metabolically active but are no longer able to reproduce. Without being bound by any theory or mechanism, it is believed that the microorganisms, when transformed, contain nucleic acid damage that renders them incapable of reproducing but still having an intact cell wall that allows them to remain temporarily viable. In many instances, these altered microorganisms can be externally indistinguishable from unaltered microorganisms, thereby making it difficult to determine how effectively a biological contamination has been addressed until time-consuming culturing tests have been completed. In addition, while still living, the altered microorganisms can still cause detrimental effects, including those noted above.

### SUMMARY OF THE INVENTION

The present invention generally relates to the monitoring of microorganisms, and, more specifically, to the use of one or more integrated computational elements to determine the effectiveness of a microorganism treatment.

In some embodiments, the present invention provides a method comprising: providing a substance comprising a plurality of viable microorganisms; exposing the substance to a light source for a sufficient length of time to form at least some non-viable microorganisms; and determining a microorganism load of the substance using one or more integrated computational elements.

In some embodiments, the present invention provides a method comprising: measuring viable microorganisms in a substance, identifying one or more types of microorganisms in a substance, or any combination thereof using one or more integrated computational elements; after measuring viable microorganisms or identifying one or more types of microorganisms in the substance, exposing the substance to a pulsed light source operable for rendering at least a portion of the microorganisms non-viable; and after or while exposing the substance to the pulsed light source, determining a microorganism load of the substance using one or more integrated computational elements.

In some embodiments, the present invention provides a device comprising: a pulsed light source configured to expose a substance to electromagnetic radiation suitable for rendering one or more microorganisms non-viable; and one or more integrated computational elements configured for determining a microorganism load of the substance after or during its exposure to the pulsed light source.

The features and advantages of the present invention will be readily apparent to one having ordinary skill in the art upon a reading of the description of the preferred embodiments that follows.

### BRIEF DESCRIPTION OF THE DRAWING

The following FIGURE is included to illustrate certain aspects of the present invention, and should not be viewed as an exclusive embodiment. The subject matter disclosed is capable of considerable modification, alteration, and equivalents in form and function, as will occur to one having ordinary skill in the art and the benefit of this disclosure.

FIG. 1 shows a schematic of an illustrative integrated computational element (ICE).

### DETAILED DESCRIPTION

The present invention generally relates to the monitoring of microorganisms, and, more specifically, to the use of one or more integrated computational elements to determine the effectiveness of a microorganism treatment.

As discussed above, conventional methods for monitoring and addressing biological contamination may be limited both by their effectiveness and timeliness of producing results. In order to address the foregoing issues and others, devices and methods described herein have been developed that may enhance the effectiveness of remediating biological contamination and enable rapid determination of a treatment's effectiveness. In particular, the devices and methods described herein utilize a pulsed light source in combination with one or more integrated computational elements to accomplish the foregoing. The pulsed light source may result in a more effective remediation of microorganisms than do continuous-operation (i.e., non-pulsed) light sources, as discussed hereinafter. In addition, detection and analysis of microorganisms using one or more integrated computational elements may take place much more rapidly than through conventional biological assays, such as culturing and PCR.

The methods and devices described herein may be used in any field where it is desirable to assay for microorganisms and/or determine the effectiveness of a remediation operation used to control microorganisms. Given the benefit of the present disclosure, one having ordinary skill in the art will be able to apply the techniques described herein to any application in which it is desirable to control and measure microorganisms in a substance. Without limitation, the methods and devices described herein may be used in fields such as, for example; water analyses, including drinking water, waste water, and processing water analyses; foodstuff, beverage, pharmaceutical, and cosmetic analyses; surface analyses; oil, gas, treatment fluid, drilling mud, and subterranean fluid analyses; and the like. In addition, the methods and devices described herein may be used in the healthcare industry to assay for biological contamination on surfaces such as, for example medical devices, surgical instruments, and the like. Other industries where it may be desirable to monitor for biological contamination on a surface may be envisioned by one having ordinary skill in the art.

In contrast to continuous-operation light sources, pulsed light sources deliver short bursts of highly intense electromagnetic radiation to a substance to address biological contamination therein. It is believed that pulsed electromagnetic radiation is often much more damaging to biological materials than is non-pulsed electromagnetic radiation and may result in more effective biological remediation of a substance. Without being bound by any theory or mechanism, it is believed that pulsed light sources, in contrast to continuous-operation light sources, may reduce the integrity of microorganisms' cell walls, thereby resulting in significantly increased outright killing of the microorganisms to render them non-viable. By achieving outright killing of the microorganisms, one may eliminate the possibility of the microorganism being able to cause further issues while still remaining viable, such as illness, turbidity, and biofouling, for example.

As disclosed in commonly owned U.S. patent application Ser. No. 13/204,294, filed on Aug. 5, 2011 and incorporated herein by reference in its entirety, one or more integrated computational elements may be used to rapidly detect and analyze particular types of bacteria, including whether the bacteria are living or dead. Those techniques may be extended to other types of microorganisms, as discussed hereinafter. Microorganism analyses may be conducted using one or more integrated computational elements much more rapidly than with conventional biological assays. The rapidity by which integrated computational elements may perform analyses is advantageous for a number of applications, and it is particularly advantageous for analyses of biological materials, including microorganisms.

The rapid analyses offered by integrated computational elements may be particularly advantageous when used to analyze bioremediation that has been conducted using a pulsed light source. Specifically, the integrated computational elements may be used to assess the degree to which microorganisms or classes of microorganisms have been rendered non-viable by a pulsed light treatment. As noted above, use of a pulsed light source may result in significantly increased outright killing of microorganisms through cell wall integrity disruption relative to inactivation through nucleic acid damage, although the present methods are not limited to these or other mechanisms of action. Differentiation between viable microorganisms and inactivated microorganisms may be difficult to detect, and it may be problematic to determine the effectiveness of a biological remediation until culturing or a related technique has taken place to determine viability. However, differentiation between viable microorganisms and non-viable microorganisms that have been killed outright through cell wall modifications may be determined readily using one or more integrated computational elements, as described herein. For example, in some embodiments, the foregoing may be accomplished by using an integrated computational element that is configured for detecting the original viable microorganisms and an integrated computational element that is configured for detecting non-viable microorganisms that have been altered by altering their cell walls. To measure an amount of microorganisms in the substance, an output of the integrated computational element may be correlated with a concentration of the microorganisms in a substance.

Due to the rapidity at which integrated computational elements may provide information about a population of microorganisms, they may be used advantageously for conducting real-time or near real-time biological analyses, thereby satisfying an unmet need in the art. Furthermore, they may be used to follow and proactively manage the progress of a biological remediation operation (e.g., using a pulsed light source) in real-time or near-real time. That is, feedback from analyses conducted using one or more integrated computational elements may be used to alter a biological remediation operation, particularly one conducted using a pulsed light source, in order to improve its effectiveness. For example, if an analysis indicates that unacceptably high levels of viable microorganisms remain in a substance during or following its exposure to a pulsed light source, the operational parameters associated with the pulsed light source may be altered in an attempt to increase the treatment effectiveness (e.g., different pulse lengths, pulse intensities, pulse sequences, pulse waveforms, number of pulses, total exposure time, combinations thereof, and the like), or a different pulsed light source may be used if a particular one is not producing a desired effect. Thus, the combination of a pulsed light source and one or more integrated computational elements for treatment feedback may be used to more effectively conduct biological remediations of a substance. Conventional microorganism assay techniques, in contrast, are simply too slow to allow proactive management of biological remediation operations to take place.

As used herein, the term “electromagnetic radiation” refers to radio waves, microwave radiation, infrared and near-infrared radiation, visible light, ultraviolet radiation, X-ray radiation and gamma ray radiation.

As used herein, the term “light source” refers to a device that emits electromagnetic radiation. Thus, as used herein, light sources are not limited to devices that only emit visible light. In some embodiments, light sources may be monochromatic, emitting substantially only a single wavelength. In other embodiments, light sources may be polychromatic, emitting a plurality of wavelengths, which may comprise a range of wavelengths.

As used herein, the term “continuous-operation light source” refers to a light source that continually produces electromagnetic radiation of substantially the same output intensity.

As used herein, the term “pulsed light source” refers to a light source that produces electromagnetic radiation not having the same output intensity at all times. In some embodiments, a pulsed light source may be cycled between a high intensity ON state and an OFF state. In some or other embodiments, a pulsed light source may be cycled between a high intensity first state and a low intensity second state, where the low intensity state does not represent a state where the pulsed light source is completely turned OFF.

As used herein, the term “microorganism” refers to a unicellular or multi-cellular microscopic life form. Microorganisms may include, but are not limited to, bacteria, protobacteria, protozoa, phytoplankton, viruses, fungi, and alga. It is to be recognized that some microorganisms may be large enough to be seen with the naked eye.

As used herein, the term “viable microorganisms” refers to microorganisms that are substantially unaltered from their native state and are capable of normal metabolic activity, including reproduction. As used herein, the term “non-viable microorganisms” refers to microorganisms that are no longer metabolically active. In some embodiments, non-viable microorganisms may refer to microorganisms that have had their cell wall ruptured, degraded, or modified by exposure to a degradative agent, such as a pulsed light source, for example. As used herein, the term “inactivated microorganisms” refers to microorganisms that have been altered from their native state and are no longer capable of reproducing. The alteration to form inactivated microorganisms may be temporary or permanent. Permanent alterations may include nucleic acid mutations, for example. Temporary alterations may include, for example, environmental conditions (e.g., temperature or lack of an appropriate nutrient source) that impact the microorganism's ability to reproduce or otherwise perform normal metabolic functions, but from which the microorganism may recover once returned to more favorable conditions. In the case of viruses, the term “viable viruses” refers to viruses that are capable of infecting host cells and replicating therein, and the term “non-viable viruses” refers to viruses that are incapable of replicating in host cells.

As used herein, the term “microorganism load” refers to the type and/or quantity of viable microorganisms and/or non-viable microorganisms in a substance.

As used herein, the term “substance” and variations thereof refer to any fluid or any solid substance or material. Solid substances or materials may include, but are not limited to, rock formations, concrete, metal, plastic, and the like.

As used herein, the term “fluid” refers to any substance that is capable of flowing, including particulate solids, liquids, gases, slurries, emulsions, powders, muds, glasses, any combination thereof, and the like. In some embodiments, the fluid can comprise an aqueous fluid, including water, mixtures of water and water-miscible fluids, and the like. In some or other embodiments, the fluid can comprise an oleaginous fluid or like hydrocarbon-based fluid.

As used herein, the terms “real-time” and “near real-time” refer to an analysis of a substance that takes place in substantially the same time frame as the interrogation of the substance with electromagnetic radiation.

Without being bound by any particular theory or mechanism, it is believed that analyses of viable microorganisms and non-viable microorganisms in a substance may be based upon monitoring of internal structures within the microorganisms. As discussed above, irradiating microorganisms with a high intensity pulsed light source may result in increased cell wall integrity disruption, thereby exposing the microorganisms' internal structures (e.g., nucleus, ribosomes, endoplasmic reticulum, and the like). Once exposed, the internal structures may be in a significantly different chemical environment that can be detected using one or more integrated computational elements. In some embodiments, a dye or like tracer that interacts with the exposed internal structures may be used to further enhance their detectability. Specifically, when low quantities of the internal structures are present or they are only weakly spectrally active, one or more integrated computational elements configured to detect a complex between a dye or like tracer and an internal structure may be used. In some or other embodiments, detection of viable microorganisms and non-viable microorganisms may be based upon detection of the changes that occur in their cell walls after being exposed to a pulsed light source. When detecting viable microorganisms and non-viable microorganisms by cell wall alterations, one or more integrated computational elements may be used that are configured to detect and analyze unaltered and altered cell walls.

As discussed briefly above, it is believed that the types of microorganisms applicable for analysis by the present methods are not particularly limited. Again without being bound by any theory or mechanism, it is believed that determination of the remediation effectiveness for a particular type of microorganism following its exposure to a pulsed light source may be based upon morphological changes that can be detected and quantified using one or more integrated computational elements. Detection of the changes induced in microorganisms following their exposure to a pulsed light source may be based upon any of the techniques described above, for example. Since the morphological changes that occur following exposure to a pulsed light source may be substantially similar across various microorganism types, it is believed that any type of microorganism may be analyzed by the methods described herein. In various embodiments, the microorganisms being detected may comprise at least one type of microorganism selected from the group consisting of bacteria (including aerobic bacteria and anerobic bacteria), protobacteria, protozoa, phytoplankton, viruses, fungi, alga, and any combination thereof. Particular classes of bacteria that may be of interest include, for example, gram-positive and gram-negative bacteria, aerobic and anaerobic bacteria, sulfate-reducing bacteria, nitrate-reducing bacteria, or any combination thereof. In some embodiments, bacteria of genera such as, for example, Y-proteobacteria, α-proteobacteria, δ-proteobacteria, Clostridia, Methanohalophilus, Methanoplanus, Methanolobus, Methanocalculus, Methanosarcinaceae, Halanaerobium, Desulfobacter, Marinobacter, Halothiobacillus, and Fusibacter may be detected and analyzed by the techniques described herein. In more specific embodiments, bacteria of interest in the oilfield industry that may be detected and analyzed using the methods described herein include, for example, Desulfovibrio desulfuricans, Desulfovibrio vulgaris, Desulfosarcina variabilis, Desulfobacter hydrogenophilus, Bdellovibrio bacteriovorus, Myxococcus xanthus, Bacillus subtilis, and Methanococcus vannielii.

In some embodiments, methods described herein can comprise: providing a substance comprising a plurality of viable microorganisms; exposing the substance to a light source for a sufficient length of time to form at least some non-viable microorganisms; and determining a microorganism load of the substance using one or more integrated computational elements. During the length of time they are being exposed, the microorganisms may absorb a sufficient amount of electromagnetic radiation to render them non-viable. In some embodiments, the microorganism load may be determined after or while exposing the substance to the light source. In some embodiments, the microorganism load may be determined before exposing the substance to the light source. In still other embodiments, the microorganism load may be determined both before and after exposing the substance to the light source.

In some embodiments, the light source may comprise a pulsed light source. In some embodiments, the pulsed light source may comprise a pulsed ultraviolet (UV) light source. In some embodiments, the light source may comprise a continuous-operation light source, such as a mercury vapor UV light source, for example. In still other embodiments, the light source may comprise a combination of a continuous-operation light source and a pulsed light source. For example, in some embodiments, a pulsed UV light source may be used in combination with a continuous-operation mercury vapor UV light source. Use of the combination of a continuous-operation light source and a pulsed light source may be advantageous for producing microorganism activation by multiple mechanisms (e.g., via genetic damage and cell wall integrity disruption). When used, the substance may be exposed to the continuous-operation light source before, after, or while exposing the substance to the pulsed light source.

In some embodiments, the pulsed light source may be switched OFF between pulses. In other embodiments, the pulsed light source may cycle between a first state where it produces a high intensity output of electromagnetic radiation and a second state where it produces a low intensity output of electromagnetic radiation, but the light source is not switched OFF between pulses. In still other embodiments, a waveform of the pulses produced by the pulsed light source may be controlled, such that at least some of the pulses differ in pulse length or pulse intensity from other pulses.

In some embodiments, methods described herein may comprise: measuring viable microorganisms in a substance, identifying one or more types of microorganisms in a substance, or any combination thereof using one or more integrated computational elements; after measuring viable microorganisms or identifying one or more types of microorganisms in the substance, exposing the substance to a pulsed light source operable for forming rendering at least a portion of the microorganisms non-viable; and after or while exposing the substance to the pulsed light source, determining a microorganism load of the substance using one or more integrated computational elements.

The underlying theory behind using integrated computational elements for conducting analyses is described in more detail in the following commonly owned U.S. patents and patent application Publications, each of which is incorporated herein by reference in its entirety: U.S. Pat. Nos. 6,198,531, 6,529,276, 7,123,844, 7,834,999, 7,911,605, 7,920,258, 2009/0219538, 2009/0219539, and 2009/0073433. Accordingly, the theoretical aspects of integrated computational elements will not be discussed in any great detail herein.

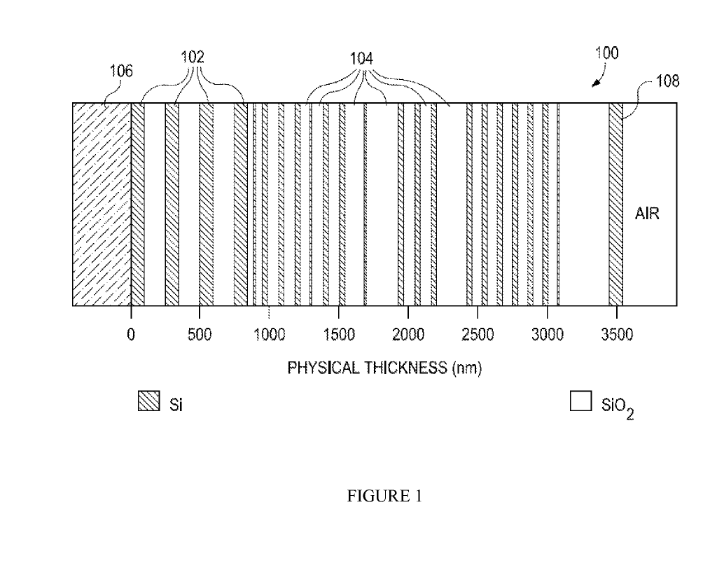

FIG. 1 shows a schematic of an illustrative integrated computational element (ICE) 100. As illustrated in FIG. 1, ICE 100 may include a plurality of alternating layers 102 and 104 of varying thicknesses disposed on optical substrate 106. In general, the materials forming layers 102 and 104 have indices of refraction that differ (i.e., one has a low index of refraction and the other has a high index of refraction), such as Si and SiO₂. Other suitable materials for layers 102 and 104 may include, but are not limited to, niobia and niobium, germanium and germania, MgF, and SiO. Additional pairs of materials having high and low indices of refraction can be envisioned by one having ordinary skill in the art, and the composition of layers 102 and 104 is not considered to be particularly limited. In some embodiments, the material within layers 102 and 104 can be doped, or two or more materials can be combined in a manner to achieve a desired optical response. In addition to solids, ICE 100 may also contain liquids (e.g., water) and/or gases, optionally in combination with solids, in order to produce a desired optical response. The material forming optical substrate 106 is not considered to be particularly limited and may comprise, for example, BK-7 optical glass, quartz, sapphire, silicon, germanium, zinc selenide, zinc sulfide, various polymers (e.g., polycarbonates, polymethylmethacrylate, polyvinylchloride, and the like), diamond, ceramics, and the like. Opposite to optical substrate 106, ICE 100 may include layer 108 that is generally exposed to the environment of the device or installation in which it is used.

The number, thickness, and spacing of layers 102 and 104 may be determined using a variety of approximation methods based upon a conventional spectroscopic measurement of a sample. These methods may include, for example, inverse Fourier transform (IFT) of the optical transmission spectrum and structuring ICE 100 as a physical representation of the IFT. The approximation methods convert the IFT into a structure based on known materials with constant refractive indices.

It should be understood that illustrative ICE 100 of FIG. 1 has been presented for purposes of illustration only. Thus, it is not implied that ICE 100 is predictive for any particular type of viable or non-viable microorganism. Furthermore, it is to be understood that layers 102 and 104 are not necessarily drawn to scale and should therefore not be considered as limiting of the present disclosure. Moreover, one having ordinary skill in the art will readily recognize that the materials comprising layers 102 and 104 may vary depending on factors such as, for example, the types of microorganisms being analyzed and the ability to accurately conduct their analysis, cost of goods, and/or chemical compatibility issues.

In addition, significant benefits can often be realized by combining the outputs of two or more integrated computational elements with one another when analyzing a single constituent or characteristic of interest. Specifically, significantly increased detection accuracy may be realized. Analysis techniques and devices utilizing combinations of two or more integrated computational elements are described in commonly owned U.S. patent application Ser. Nos. 13/456,255, 13/456,264, 13/456,283, 13/456,302, 13/456,327, 13/456,350, 13/456,379, 13/456,405, and 13/456,443, each filed on Apr. 26, 2012 and hereby incorporated by reference in their entireties.

To detect and analyze microorganisms using an integrated computational element, a substance containing the microorganisms may be illuminated with a source of electromagnetic radiation during or after its exposure to the pulsed light source. Suitable sources of electromagnetic radiation may include, for example, infrared (including near-infrared) radiation, visible light, and/or ultraviolet radiation. In some embodiments, the substance may be stimulated to emit electromagnetic radiation upon its illumination with the source of electromagnetic radiation (e.g., fluorescent emission and/or phosphorescent emission). In some embodiments, the electromagnetic radiation may optically interact with the substance and then optically interact with the integrated computational element (e.g., via transmission, reflection, transflection, or through the use of evanescent radiation). In other embodiments, the electromagnetic radiation may optically interact with the integrated computational element and then optically interact with the substance. In either instance, following optical interaction with the integrated computational element and the substance, the electromagnetic radiation may be received by a detector. The output of the detector may then be correlated with a property of the substance, such as the substance's microorganism load. As used herein, the term “optically interact” and variants thereof refer to the reflection, transmission, scattering, diffraction, or absorption of electromagnetic radiation by a substance or an integrated computational element.

A wide variety of pulsed light sources may be suitable for use in the embodiments described herein. In some embodiments, the pulsed light source may produce electromagnetic radiation pulses of substantially a single wavelength (i.e., a monochromatic or substantially monochromatic source of electromagnetic radiation, which may also be coherent, such as a laser or light-emitting diode, for example). In other embodiments, the pulsed light source may be polychromatic and produce electromagnetic radiation pulses exhibiting a range or plurality of wavelengths. In some embodiments, the pulsed light source may comprise a pulsed ultraviolet light source. In some embodiments, suitable pulsed light sources may produce at least a wavelength range of about 100 nm to about 280 nm. As one of ordinary skill in the art will recognize, this wavelength range encompasses the ultraviolet C (UVC) range, which can be very effective for rendering microorganisms non-viable. Pulsed light sources producing other wavelength ranges may also be effective. In some embodiments, suitable pulsed light sources may produce at least a wavelength range of about 10 nm to about 100 nm, or about 280 nm to about 315 nm (ultraviolet B), or about 315 nm to about 400 nm (ultraviolet A), or about 400 nm to about 700 nm (visible light). In some embodiments, a broad spectrum pulsed light source producing an output of electromagnetic radiation from about 100 nm to about 700 nm or from about 100 nm to about 800 nm may be used. Other suitable types of electromagnetic radiation that may be used in the pulsed light source include, for example, radio waves, microwave radiation, infrared and near-infrared radiation, visible light, vacuum ultraviolet radiation, X-ray radiation and gamma ray radiation.

In some embodiments, the pulsed light source may comprise a pulsed xenon or pulsed krypton ultraviolet light source. That is, the pulsed light source may produce electromagnetic radiation generated by excitation of these gases. Pulsed xenon ultraviolet light, for example, may be characterized as a polychromatic broad band emission having a wavelength range of about 180 nm to about 800 nm. As a non-limiting example, a pulsed xenon ultraviolet light source may deliver energy to a substance at a rate of about 10 Joules/second through emission of pulses having a power of approximately 1000 watts, a pulse length of about 10 ms, and a frequency of about 10 s⁻¹. For comparison, a continuous-operation mercury vapor ultraviolet light source also delivering energy to a substance at a rate of about 10 Joules/second produces only approximately a 10 watt continuous emission.

In some embodiments, the pulsed light source may comprise a mercury vapor pulsed light source. As one of ordinary skill in the art will recognize, mercury vapor ultraviolet light sources typically produce more discrete emission bands than do pulsed xenon or pulsed krypton ultraviolet light sources, although the latter ultraviolet light sources may be more intense.

In addition to the wavelength range and type of electromagnetic radiation, operational parameters that may be varied for the pulsed light source include, for example, the light intensity (i.e., the energy density), the pulse frequency, the pulse width, the pulse waveform and variations thereof, the exposure time to a substance, or any combination thereof. In some embodiments, a combination of short and long pulses may be used, optionally varying the pulse intensity. In some or other embodiments, a combination of short, medium, and long pulses or more complex pulse sequences may be used, optionally varying the pulse intensity.

In some embodiments, suitable pulsed light sources may produce light intensities ranging between about 0.01 J/cm² and about 50 J/cm². In some embodiments, suitable pulsed light sources may produce light intensities ranging between about 0.1 J/cm² and about 50 J/cm². Continuous-operation light sources, in contrast, may not be amenable to ongoing operation at the much higher light intensities that can be achieved with pulsed light sources. In some embodiments, suitable pulsed light sources may produce pulse frequencies ranging between about 1 s⁻¹ and about 10,000 s⁻¹ or between about 5 s⁻¹ and about 30 s⁻¹. In some embodiments, suitable pulsed light sources may produce pulse widths ranging between about 0.1 ns and about 100 s, or between about 0.1 μs and about 100 μs, or between about 1 ms and about 1 s. In some embodiments, suitable exposure times of the pulsed light source to the substance may range between about 0.1 s and about 60 minutes, or between about 1 s and about 15 minutes, or between about 10 s and about 10 minutes. In some embodiments, during the exposure time, sufficient energy may be supplied to the microorganisms to at least partially render them non-viable.

In some embodiments, determining a microorganism load of the substance using one or more integrated computational elements may comprise measuring viable microorganisms in the substance, measuring non-viable microorganisms in the substance, identifying one or more types of microorganisms in the substance, or any combination thereof. For example, when analyzing bacteria, determining a microorganism load may comprise measuring viable bacteria in the substance, measuring non-viable bacteria in the substance, determining one or more types of bacteria in the substance, or any combination thereof. In some embodiments, the methods described herein may comprise detecting a particular subclass of microorganisms (e.g., aerobic and anaerobic bacteria) and/or how effective a pulsed light source has been in rendering them non-viable. In some embodiments, the methods may comprise detecting a specific type (i.e., species) of microorganism and/or how effective a pulsed light source has been in rendering it non-viable. As one of ordinary skill in the art will recognize, some particular types or species of microorganisms may be more problematic than others in a given application, and their remediation may be more of a concern than others. For example, anaerobic bacteria may produce hydrogen sulfide as a metabolic product, which may be very undesirable for a number of applications, including subterranean operations, due to corrosion and toxicity issues. Thus, the ability to rapidly determine the viability of particular types or species of microorganisms using the present methods may be particularly advantageous.

In some embodiments, determining a microorganism load of the substance may take place in real-time or near real-time while the substance is being exposed to the pulsed light source. In other embodiments, determining a microorganism load of the substance may take place after exposure to the pulsed light source takes place. That is, in some embodiments, determining a microorganism load of the substance may take place offline in non-real-time. In still other embodiments, the present methods may be used to follow a microorganism load of a substance with time. For example, the kinetic growth or decline of a microorganism population may be followed using one or more integrated computational elements.

In some embodiments, the methods described herein may be used to determine the microorganism load of a substance and the changes in the microorganism load brought about through exposure to a pulsed light source. That is, in some embodiments, the present methods may be used to determine the effectiveness of a pulsed light treatment for remediation of a microorganism contamination. In some embodiments, the methods described herein may be used to determine the microorganism load of the substance before and after its exposure to a pulsed light source. For example, in some embodiments, the methods may further comprise measuring the viable microorganisms in the substance using an integrated computational element, before exposing the substance to the pulsed light source. Thus, in such embodiments, decreases in viable microorganisms following exposure to a pulsed light source may be observed.

By evaluating the effectiveness of a pulsed light treatment upon microorganism levels, future treatments may be better designed to improve their effectiveness and/or treatment of a substance may be repeated and optionally modified to reduce microorganism levels to a suitable level. In some embodiments, the methods described herein may further comprise adjusting one or more operational parameters associated with the pulsed light source in response to the microorganism load determined for the substance using the one or more integrated computational elements. Operational parameters of the pulsed light source that may be altered in response to the determined microorganism load may include, for example, the light intensity, the pulse width, the pulse frequency, the wavelength range, exposure time to the pulsed light source, the pulse waveform, the variety of pulse waveforms employed, the bias light intensity, or any combination thereof. As used herein, the term “bias light intensity” refers to a low intensity state in a pulsed light source that is not cycled completely to an OFF state following delivery of high intensity pulse of electromagnetic radiation. In some embodiments, the operational parameter(s) may be adjusted in real-time or near real-time while measuring the microorganism load of the substance. In some embodiments, the present methods may further comprise repeating the exposure of the substance to the pulsed light source following alteration of the pulsed light source's operational parameter(s).

In still other embodiments, the present methods may further comprise use of a biocide in conjunction with the pulsed light source. That is, in some embodiments, the methods may further comprise introducing a biocide to the substance being treated with the pulsed light source. Biocides suitable for use with particular microorganisms will be familiar to one having ordinary skill in the art. Use of a biocide in conjunction with the pulsed light source may further improve the production of non-viable microorganisms. For example, the biocide may weaken the microorganisms and make them more susceptible to inactivation with the pulsed light source. In the alternative, a biocide may target a population of microorganisms not targeted by the pulsed light treatment, or a biocide may be more effective after the microorganisms are weakened by exposure to a pulsed light source. In some embodiments, the microorganisms may be exposed to the biocide prior to being exposed to the pulsed light source. In other embodiments, the microorganisms may be exposed to the biocide while being exposed to the pulsed light source. In still other embodiments, the microorganisms may be exposed to the biocide after being exposed to the pulsed light source. The integrated computational element may be used to analyze the microorganisms at any point during this process.

In some embodiments, the methods described herein can further comprise determining a kill ratio for a population of microorganisms that has been exposed to a pulsed light source. As used herein, the term “kill ratio” refers to the number of non-viable microorganisms present in a substance after exposure to a pulsed light source relative to the number of viable microorganisms present in the substance before exposure. The kill ratio can be determined, in some embodiments, by quantifying the viable microorganisms before and after exposure to a pulsed light source takes place. In other embodiments, non-viable microorganisms may be determined instead. In some embodiments, the kill ratio can be at least about 75%. In other embodiments, the kill ratio can be at least about 80%, or at least about 85%, or at least about 90%, or at least about 95%, or at least about 96%, or at least about 97%, or at least about 98%, or at least about 99%. In some embodiments, if a desired kill ratio is not attained, the methods can further comprise repeating exposure to the pulsed light source, as described above, changing one or more operational parameters of the pulsed light source and/or continuous-operation light source, if used, or performing a different remediation treatment for controlling the microorganisms (e.g., a biocidal treatment).

The types of substances that may be treated using a pulsed light source and analyzed for microorganisms using the present methods are not believed to be particularly limited. In some embodiments, the substance may comprise a fluid. In other embodiments, the substance may comprise a solid surface. In some embodiments, the fluid or solid surface may be substantially opaque to visible light. As one of ordinary skill in the art will recognize, these types of substances may sometimes be less effectively treated with visible pulsed light treatments. However, by choosing a wavelength or wavelength range of electromagnetic radiation where the fluid or solid surface is more transparent to the electromagnetic radiation, an effective pulsed light treatment may still be realized. For example, oil, which is substantially opaque in the visible region, may be substantially transparent to near-infrared electromagnetic radiation. In addition, one having ordinary skill in the art will recognize that pulsed light sources may sometimes create short term transmission paths for electromagnetic radiation through a substance through the creation of various excited electronic states. Thus, in some embodiments, a first pulse of electromagnetic radiation may be used to create a substance in an excited electronic state that is then transparent to a second pulse of electromagnetic radiation, in order to effectively treat a substance with pulsed light. In some or other embodiments, a continuous-operation light source may be used to improve the transparency of an opaque substance to electromagnetic radiation in a manner similar to that described above.

As one of ordinary skill in the art will recognize, solid surfaces may be particularly susceptible to growth of microorganisms thereon. One of ordinary skill in the art will further recognize that microorganism contamination upon a surface may result in a number of deleterious effects including, for example, biofouling, permeability reduction, structural failure, corrosion, health hazards, and any combination thereof. Contamination by microorganisms can be particularly problematic in a pipeline or like fluid conduit. In a pipeline or like fluid conduit, microorganisms can sometimes aggregate in joints, welds, seams, and the like, where they may significantly increase the risk of structural failure. As noted above, anaerobic bacteria may be particularly problematic in this regard due to the hydrogen sulfide that they produce as a metabolic byproduct. The methods described herein may be used to reduce the deleterious effects associated with microorganism contamination. Solid surfaces that may be exposed to a pulsed light source and analyzed by the methods described herein include, for example, pipeline surfaces, welds, proppant particulates, subterranean formation surfaces, wellbore surfaces, medical device and surgical instrument surfaces, food preparation surfaces, reactor vessel surfaces, and the like.

In some embodiments, the fluid being exposed to a pulsed light source and analyzed by the present methods may comprise an aqueous fluid such as water. In some or other embodiments, the fluid may comprise an oleaginous fluid, such as oil or a hydrocarbon. In some embodiments, the fluid may be static (i.e., not moving) while being analyzed by the methods described herein. In other embodiments, the fluid may be in motion while being analyzed. In some embodiments, determining a microorganism load of the substance may take place while the fluid is flowing (e.g., in a pipeline or like fluid conduit). In some embodiments, the fluid may be flowing while being exposed to the pulsed light source.

Water used in subterranean operations can sometimes be obtained from a number of “dirty” water sources, having varying levels of bacterial or other types of microorganism contamination therein. Although microorganism contamination may sometimes not be particularly problematic at ambient temperatures on the earth's surface, once the water is introduced into a more favorable growth environment, microorganism levels and their detrimental effects may rapidly increase. For example, when introduced into a warm subterranean environment, even low levels of microorganisms can multiply quickly and potentially lead to damage of a subterranean formation. Likewise, favorable growth conditions may sometimes be found in a pipeline or like fluid conduit. In some cases, microorganisms may lead to biofouling of a subterranean surface or pipeline surface. As discussed above, anaerobic bacteria may be particularly detrimental when introduced into a subterranean formation or a pipeline due to the hydrogen sulfide that they produce. In some cases, aerobic bacteria may be tolerable, at least to some extent. Rapidly multiplying microorganisms and their metabolic byproducts can quickly clog and corrode production tubulars, plug formation fractures, and/or produce hydrogen sulfide which represents a health hazard and can lead to completion failure and loss of production. Accordingly, it can be highly desirable to reduce microorganism levels before or while introducing a fluid to a subterranean formation.

In some embodiments, the methods described herein may further comprise introducing a fluid into a subterranean formation, such as via a wellbore.

In some embodiments, the fluid may comprise a treatment fluid or a drilling mud. As used herein, the term “treatment fluid” refers to a fluid that is placed in a location (e.g., in a subterranean formation or in a pipeline or like fluid conduit) in order to perform a desired function or to achieve a desired purpose. Treatment fluids can be used in a variety of subterranean operations, including, but not limited to, drilling operations, production operations, stimulation operations, remediation operations, fluid diversion operations, secondary or tertiary enhanced oil recovery (EOR) operations, and the like. As used herein, the terms “treat,” “treatment,” “treating,” and other grammatical equivalents thereof refer to any operation that uses a fluid in conjunction with performing a desired function and/or achieving a desired purpose. The terms “treat,” “treatment,” and “treating,” as used herein, do not imply any particular action by the fluid or any particular component thereof unless otherwise specified. Treatment fluids can include, for example, fracturing fluids, acidizing fluids, conformance treatment fluids, damage control fluids, remediation fluids, scale removal and inhibition fluids, chemical floods, and the like. In some embodiments, the treatment fluid or drilling mud may be exposed to a pulsed light source and analyzed using one or more integrated computational elements prior to being introduced to the subterranean formation. In some or other embodiments, the treatment fluid or drilling mud may be exposed to a pulsed light source and analyzed using one or more integrated computational elements while being introduced to the subterranean formation. In still other embodiments, the treatment fluid or drilling mud may be exposed to the pulsed light source and analyzed using one or more integrated computational elements while in the subterranean formation. In some embodiments, the present methods may be used, at least in part, to render a produced fluid, such as a produced formation fluid or a produced treatment fluid, suitable for being re-introduced to a subterranean formation.

In some embodiments, the methods described herein can further comprise determining suitable operational parameters for a pulsed light source used in conjunction with remediating microorganism contamination in a substance. By determining the number and/or types of microorganisms present in a substance before exposing the substance to a pulsed light source, more suitable operational parameters to address the particular type and/or extent of microorganism contamination may be used. For example, in some embodiments, the exposure time of the substance to the pulsed light source may be changed and/or the pulse width, frequency, waveform, intensity and/or cycle may be altered in response to particular quantities and/or types of microorganisms present in the substance. In some embodiments, determining suitable conditions for the pulsed light exposure may take place automatically under computer control. For example, a computer may select appropriate operational parameters for the pulsed light source based upon input data of how effectively substances having similar microorganism loads have been remediated using a pulsed light source. In other embodiments, determining suitable operational parameters for the pulsed light source may take place manually under the direction of an operator. In either case, determining suitable operational parameters for the pulsed light source may be impacted, at least in part, by the location in which the substance will be used. For example, if used under conditions not overly conducive to microorganism growth, higher microorganism levels may be somewhat more tolerable in a substance. One of ordinary skill in the art will recognize suitable levels of microorganisms that may ordinarily be present in a given application.

In some embodiments, the methods described herein may further comprise determining if the microorganism load of a fluid is suitable for being introduced to a subterranean formation. For example, if the microorganism load of the fluid is unacceptably high, or if certain types of microorganisms are present in the fluid, the fluid may be deemed unsuitable for subterranean introduction. Knowing the microorganism load of the fluid and conditions present within a given subterranean formation, one of ordinary skill in the art will be able to determine a fluid's suitability for introduction to a given subterranean formation. In various embodiments, determination of a fluid's suitability for introduction to a particular subterranean formation may be made automatically under computer control or manually by an operator. In some embodiments, the methods may further comprise re-exposing the fluid to the pulsed light source, optionally after altering one or more operational parameters thereof, prior to or while the fluid is being introduced to the subterranean formation. In some or other embodiments, the fluid may be exposed to the pulsed light source after introduction to the subterranean formation.

In some embodiments, devices for detecting and analyzing microorganisms are described herein. In some embodiments, the devices may comprise a pulsed light source configured to expose a substance to electromagnetic radiation suitable for rendering one or more microorganisms non-viable; and one or more integrated computational elements configured for determining a microorganism load of the substance after or during its exposure to the pulsed light source. In some embodiments, the pulsed light source may comprise a pulsed ultraviolet light source. In some embodiments, the devices may further comprise a continuous-operation light source, which may comprise a mercury vapor ultraviolet light, for example. When used, the continuous-operation light source may illuminate a substance with electromagnetic radiation at the same and/or different time and/or position as the pulsed light source.

In some embodiments, the devices may be portable, such that they can be easily transported to any substance needing remediation and/or analysis of a microorganism contamination thereon. In other embodiments, the devices may be fixed in place, such as in a pipeline or tank, to provide ongoing feedback of microorganism levels present therein. In some embodiments, the devices may be configured to analyze a static substance. In other embodiments, the devices may be configured to analyze a fluid substance that is in motion.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted. the invention claimed is:

1. A method comprising:
providing a substance comprising a plurality of viable microorganisms;
exposing the substance to a light source for a sufficient length of time to form at least some non-viable microorganisms; and
determining a microorganism load of the substance using one or more integrated computational elements.

2. The method of claim 1, wherein the light source comprises a pulsed light source.

3. The method of claim 2, wherein the pulsed light source comprises a pulsed UV light source.

4. The method of claim 2, wherein the microorganism load of the substance is determined after or while exposing the substance to the pulsed light source.

5. The method of claim 4, wherein determining a microorganism load of the substance using one or more integrated computational elements comprises measuring viable microorganisms in the substance, measuring non-viable microorganisms in the substance, identifying one or more types of microorganisms in the substance, or any combination thereof.

6. The method of claim 2, further comprising:
before exposing the substance to the pulsed light source, measuring viable microorganisms in the substance, identifying one or more types of microorganisms in the substance, or any combination thereof using the one or more integrated computational elements.

7. The method of claim 2, further comprising:
introducing a biocide to the substance.

8. The method of claim 2, further comprising:
exposing the substance to a continuous-operation light source before, after, or while exposing the substance to the pulsed light source.

9. The method of claim 8, wherein the pulsed light source comprises a pulsed UV light source and the continuous-operation light source comprises a mercury vapor UV light source.

10. The method of claim 2, further comprising:
adjusting one or more operational parameters associated with the pulsed light source in response to the microorganism load determined for the substance.

11. The method of claim 2, wherein the substance comprises a fluid.

12. The method of claim 11, wherein the fluid is flowing while determining the microorganism load of the substance, exposing the substance to the pulsed light source, or both.

13. The method of claim 11, further comprising:
introducing the fluid into a subterranean formation.

14. The method of claim 2, wherein the substance comprises a solid surface.

15. The method of claim 14, wherein the solid surface comprises a fluid conduit.

16. The method of claim 2, wherein the substance comprises a drinking water, a beverage, a foodstuff, a processing water, a waste water, a pharmaceutical, a cosmetic, a medical device, an oil, a treatment fluid, a drilling mud, or any combination thereof.

17. The method of claim 2, wherein the microorganisms comprise at least one type of microorganism selected from the group consisting of aerobic bacteria, anaerobic bacteria, protobacteria, protozoa, phytoplankton, viruses, fungi, alga, and any combination thereof.

18. A method comprising:
measuring viable microorganisms in a substance, identifying one or more types of microorganisms in a substance, or any combination thereof using one or more integrated computational elements;
after measuring viable microorganisms or identifying one or more types of microorganisms in the substance, exposing the substance to a pulsed light source operable for rendering at least a portion of the microorganisms non-viable; and
after or while exposing the substance to the pulsed light source, determining a microorganism load of the substance using one or more integrated computational elements.

19. The method of claim 18, wherein the microorganisms comprise bacteria.

20. The method of claim 19, wherein determining a microorganism load of the substance comprises measuring viable bacteria in the substance, measuring non-viable bacteria in the substance, identifying one or more types or species of bacteria in the substance, or any combination thereof.

21. The method of claim 18, further comprising:
adjusting one or more operational parameters associated with the pulsed light source in response to the microorganism load determined for the substance.

22. The method of claim 18, wherein the substance comprises a solid surface.

23. The method of claim 18, wherein the substance comprises a fluid.

24. The method of claim 23, further comprising:
introducing the fluid into a subterranean formation.

25. The method of claim 18, further comprising:
introducing a biocide to the substance.

26. The method of claim 18, further comprising:
exposing the substance to a continuous-operation light source before, after, or while exposing the substance to the pulsed light source.

27. A device comprising:
a pulsed light source configured to expose a substance to electromagnetic radiation suitable for rendering one or more microorganisms non-viable; and
one or more integrated computational elements configured for determining a microorganism load of the substance after or during its exposure to the pulsed light source.
